(19) United States
(12) Patent Application Publication
(10) Pub. No.: US 2023/0098833 A1
(43) Pub. Date: Mar. 30, 2023

(54) DEEPDRUG: AN EXPERT-LED DIRECTED GRAPH NEURAL NETWORKING DRUG-REPURPOSING FRAMEWORK FOR IDENTIFICATION OF A LEAD COMBINATION OF DRUGS PROTECTING AGAINST ALZHEIMER'S DISEASE AND RELATED DISORDERS

(71) Applicants: THE UNIVERSITY OF HONG KONG, Hong Kong (CN); Ramot at Tel-Aviv University Ltd., Tel Aviv (IL)

(72) Inventors: Victor O.K. Li, Hong Kong (CN); Jacqueline C.K. Lam, Hong Kong (CN); Yang Han, Hong Kong (CN); Jocelyn Downey, Wimbledon (GB); Tushar Kaistha, Hong Kong (CN); Illana Gozes, Ramat Hasharon (IL)

(73) Assignees: THE UNIVERSITY OF HONG KONG, Hong Kong (CN); Ramot at Tel-Aviv University Ltd., Tel Aviv (IL)

(21) Appl. No.: 17/945,933

(22) Filed: Sep. 15, 2022

Related U.S. Application Data
(60) Provisional application No. 63/245,343, filed on Sep. 17, 2021.

(51) Int. Cl. G16H 20/10; A61B 5/00; G06N 5/02; G06K 9/62; A61K 31/165; A61K 31/135; A61K 31/445

(52) U.S. Cl. CPC G16H 20/10; A61B 5/4088; A61K 31/135; A61K 31/165; A61K 31/445; G06K 9/6224; G06N 5/025

(57) ABSTRACT

A novel AI-driven drug-repurposing method, DeepDrug, is used to identify a lead combination of previously FDA-approved drugs to treat AD by targeting the upstream genetic markers along the AD pathology. A three-step methodology is used. First, a heterogeneous biomedical graph is constructed comprising complex and interconnected genes, proteins, and drug information to capture the network characteristics of the AD pathology, considering the expert known associations between different AD pathways and utilizing node weighting and edge weighting and direction. Second, the curated graph is taken as an input to an artificial intelligence (AI)-driven graphical neural network (GNN) framework, with embeddings of drug and gene nodes as the outputs. Third, a drug scoring and selection analysis is conduced to generate the drug-gene scores and identify a lead combination of repurposed AD drug candidates for clinical verification.

### CROSS-REFERENCE TO RELATED APPLICATION

The present application claims the benefit of priority to U.S. Provisional Pat. Application Serial No. 63/245,343 filed Sep. 17, 2021, which is hereby incorporated herein by reference in its entirety.

### FIELD OF THE INVENTION

The present invention relates to the search for approved drugs that can be repurposed for other uses, and more particularly to the use of artificial intelligence in the search for drugs to be repurposed to treat Alzheimer’s disease.

### BACKGROUND OF THE INVENTION

Alzheimer’s disease (AD) has a significant impact on human longevity and quality of life. Globally speaking, around 50 million people suffer from AD and related forms of dementia, resulting in 28.8 million disability-adjusted life-years. [1] In general, drug development is a hugely expensive endeavour, with pharmaceutical companies spending more than $2.6 billion for the development of a single drug all the way to regulatory approval. As of today, only a handful of drugs, and one drug combination consisting of two of those drugs, have been approved by the US Food and Drug Administration (FDA) for the treatment of AD or its cognitive symptoms. Moreover, only one of the handful of approved drugs may delay clinical decline - the other drugs all treat cognitive symptoms (memory and thinking) rather than functions. To date, no effective disease-modifying treatment or preventative therapies have been found to treat AD, and the process of searching for effective AD drug candidates remains lengthy and data-constrained.

Drug-repurposing, a strategy that identifies new uses for approved drugs, has become a promising approach to drug discovery for AD and beyond. For non-AD diseases, network-based statistical methods have been proposed [2,3] to calculate the distance between drug targets and cancers, assuming that mutated genes relate to certain diseases through protein-protein interactions. [4] More recently, to combat the spread of Covid-19, artificial intelligence (AI)-driven graphical neural network (GNN) models have been successfully used to isolate drug candidates or combinations through human interactome and drug-target data. [5,6] In the context of AD, prior attempts to utilize bioinformatic methods for drug-repurposing have demonstrated the feasibility of a network-based approach using traditional statistical measures [7] and machine learning methods. [8] Using AI-driven deep learning techniques to capture the non-linearity in high-dimensional biomedical data, a GNN model has further demonstrated the potential of network-based drug-repurposing for AD. [9]

U.S. Pat. No. US 11,026,942 discloses 130 existing drugs (that have been confirmed for safety and pharmacokinetics in humans) that can be combined with each other as two or more combinations as a means for a pre-emptive treatment of people at risk of AD and in a stage before developing mild cognitive impairment (MCI). To do this, the inventors of this patent application screened an existing drug library consisting of 1280 kinds of pharmaceutical compounds approved by the Food and Drug Administration (FDA) in America by using nerve cells induced to differentiate from iPS cells derived from AD patients, and extracted 129 kinds (including one kind of concomitant drug) of compounds that protect against Aβ pathology in the nerve cells as candidate therapeutic drugs for AD. Furthermore, they have classified these candidate compounds in 10 cluster compounds based on their structure and properties and found a combination of clusters that acts additively as compared to a single agent. The inventors then conducted further studies based on these findings and completed the invention proposed in this patent application.

However, the GNN model and other models used in the past have only focussed on drugs and diseases, without considering key domain-specific knowledge, such as long genes, inflammation, immunological and aging pathways, and somatic mutation markers identified from the blood, in addition to the brain. Moreover, it remains challenging to integrate multiple datasets representative of different AD populations to identify the mutation pathways causal to AD, to determine a lead combination of candidate drugs that interact with the somatic mutation phenotypes, directly or indirectly through network-based actions.

U.S. Pat. Application Publication US 2021/0081717 A1 of Creed et al. discloses the use of a GNN model with machine learning and using biomedical information to construct a network for the prediction of drug candidates. However, because inputs to its GNN model are not provided with directions and/or weights, it lacks speed and effectiveness.

### SUMMARY OF THE INVENTION

Up till now, the underlying mechanism of AD remains largely unknown. [10] Two main important expert-led and domain-specific concepts have guided the AI-driven drug repurposing framework design of the present invention. First, a so-called “DeepDrug framework” focuses on pathways that lead to or associate with AD, and those that associate with somatic mutations, especially those found in the long genes, and those found in the blood as well as the brain. In the presence of predisposing factors, the accumulation of somatic mutations drives AD pathology, accelerated by dysregulated redox systems and DNA-repair pathways, while these somatic mutations may converge to disrupt synaptic signalling. [11] While familial AD is driven by mutations in the amyloid precursor protein (APP) or the processing enzymes, presenilin 1 and 2 (PSEN1 or PSEN2), risk factors for sporadic AD include aging, lack of education and biomarkers including the presence of the APOE-ε4 allele, tau-associated neurofibrillary-tangles, and β-amyloid plaques. Furthermore, for sporadic AD, evidence from genome-wide association studies (GWAS) data has implicated a large number of pathways associated with AD pathology, including DNA-repair pathways, redox systems, and pathways activated by established AD-associated comorbidities, such as diabetes and obesity. Simultaneously, somatic mutations accumulate with aging, augmenting in the brains of AD patients and impacting cell signalling, [12] [28] which can be most clearly observed across the long genes. Therapeutic interventions capable of compensating for dysfunctional signalling can offer high potential and valuable tools for AD treatment.

Second, the AI framework of the present invention takes a network-based approach to model somatic mutations pathway convergence and divergence and identifies a lead combination of drug candidates for AD treatment based on existing drugs that interact with dysregulated pathways in AD patients.

Based on the above ideas, an expert-led AI-driven approach, incorporating domain-specific knowledge while using relevant big genetic and drug datasets, is utilized to determine a lead combination of FDA-approved drugs that interacts with the somatic mutation phenotypes either directly or indirectly through network-based actions. This approach can significantly accelerate the process and precision of AD drug identification. This methodology is novel and is differentiated from the former drug-repurposing methodologies. First, it is an expert-led AI-driven drug-repurposing framework, incorporated with domain-specific knowledge, including knowledge about the long genes, inflammation, immunological and aging pathways, and somatic mutation markers that are identified from the blood, as well as from the brain (see FIG. 1A), and are closely associated with AD. Second, the present invention involves a heterogeneous biomedical graph, taking the expert-led knowledge into a graphical construction and weighting process, while using a network-based approach to capture the convergence and divergence of different pathways that associate with AD (see FIG. 1B). Third, the present invention uses multiple datasets representative of different AD populations (such as ADNI, ADSP etc.) to identify the key mutation pathways causal to AD, and a lead combination of drugs that target at these key causal AD pathways (see FIG. 1C).

Thus, the present invention improves on prior applications of drug-repurposing in three ways:

(1) In contrast with deepDR [9], which focused on drug and disease information, DeepDrug integrates expert-led domain-specific knowledge that encompasses the latest information of the role of long genes, immunological and aging pathways, and somatic mutation markers identified from the blood and the brain with AD.

(2) An expert-led directed heterogeneous biomedical graph is generated that encompasses a rich set of nodes and edges, carefully incorporating expert knowledge in graph construction and node/edge weighting and direction to capture crucial pathways that associate with AD. Distinguishing itself from other biomedical graph models, the present biomedical graph allows weights to be assigned to nodes and edges, and directed edges, better capturing AD domain-specific knowledge.

(3) As stated in Hsieh et al. (2020) [6], by encoding a biomedical graph through a GNN into a new embedding space, more accurate capture of the rich relationship between nodes in the original graph is possible, rather than simply relying on the shortest path metric. Building on this, the directed edges and node and edge weight assignments of the present invention, have the power to further enhance the accuracy of the prediction of successful drug candidates.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The foregoing and other objects and advantages of the present invention will become more apparent when considered in connection with the following detailed description and appended drawings in which like designations denote like elements in the various views, and wherein:

FIG. 1A is a diagram of an expert-led AI-driven drug-repurposing framework according to the present invention, FIG. 1B is a diagram of a heterogeneous biomedical graph incorporating expert-led knowledge according to the present invention and FIG. 1C is a diagram of multiple datasets used to determine a lead combination of approved drugs according to the present invention;

FIG. 2A is a diagram of heterogeneous biomedical graphs, FIG. 2B is a diagram of a graphical neural network (GNN) and FIG. 2C is a diagram of a drug scoring and selection table and lead drug combinations according to the present invention;

FIG. 3A shows the scores of 120 three-drug combinations (chosen from the top ten drugs) and FIG. 3B shows the drug targets of the five drugs involved in the top 3 three-drug combinations and the relationship between the drug targets via protein-protein interactions;

FIG. 4 is a domain-specific knowledge inspired DeepDrug framework (Key domain-specific knowledge incorporated into the model of the present invention;

FIG. 5 shows gene nodes incorporating expert-led knowledge;

FIG. 6 is a DeepDrug biomedical graph;

FIG. 7 is a graph neural network (GNN) used to embed drug and gene nodes; and

FIGS. 8A and 8B illustrate the accuracy of the GNN Model in receiver operating characteristic (ROC) and Precision Recall curves, respectively.

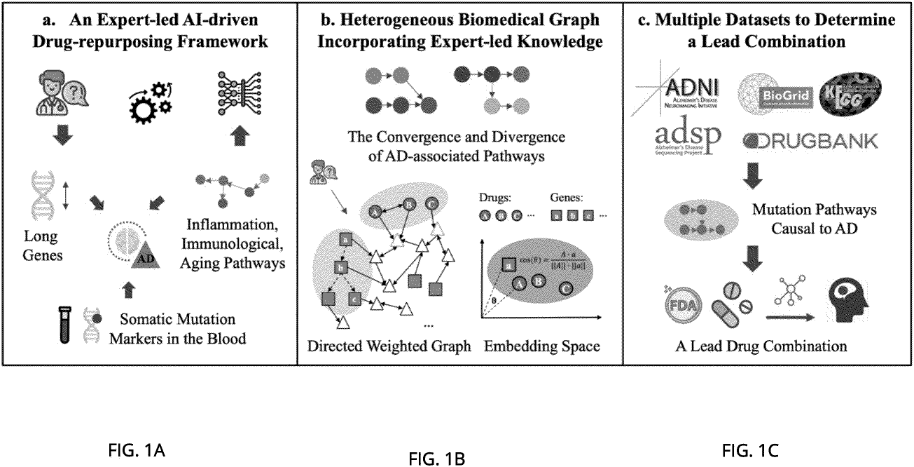

### DETAILED DESCRIPTION OF THE INVENTION

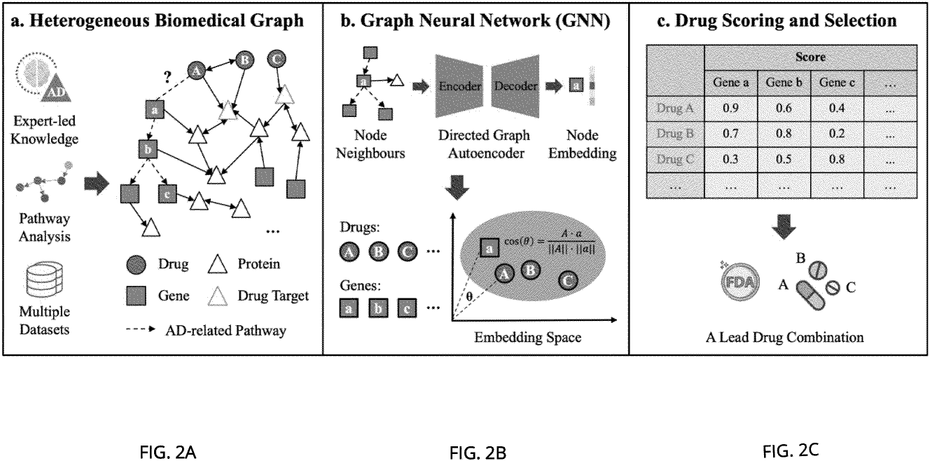

An expert-led directed GNN-based drug-repurposing framework is used to identify a lead combination of FDA-approved drugs for disease, and particularly AD treatments. The framework includes a three-step methodology. First, a heterogeneous biomedical graph consisting of complex and interconnected genes, proteins, and drug information is created to capture the network characteristics of the AD pathology, incorporating the expert-led knowledge into the graph construction and weighting process, and capturing the known associations and overlaps between different AD pathways across multiple datasets. Second, the curated graph is taken as an input to an AI-driven GNN framework, with embeddings of drug and gene nodes as the outputs. Third, a drug scoring and selection analysis is conduced to generate the drug-gene scores and identify a lead combination of repurposed AD drug candidates with the top drug-gene scores for clinical verification. FIGS. 2A-2C summarize the DeepDrug methodology of the present invention.

Based on the recent observation that long genes are more likely to be affected by somatic mutations [11], it is demonstrated below how the proposed DeepDrug framework can identify a list of top repurposed FDA-approved drugs as well as a lead combination of three repurposed drugs for AD treatment. Table 1 shows the top ten drugs and their scores according to DeepDrug. The drugs that may have negative effects on AD have been removed. The model of the present invention assesses the proximity between a drug and gene pair, since no ground truth labels can be provided during model training (i.e., that a drug can “treat” a mutation in a gene). It cannot assess the positive or negative nature of the relationship between drugs and genes, and expert knowledge is therefore required to analyze the drugs generated from the model to exclude those that will be effective drug candidates for AD. Some drugs, for example, Ziprasidone and Cannabidiol, are useful for treating AD because they have formerly been approved to treat brain diseases including Schizophrenia and seizures. Some drugs carry a positive effect on AD as they have been used for other diseases such as Paget’s disease and Osteoarthritis, which are related to aging and are commonly seen in the elderly. Other drugs such as Rosiglitazone and Nateglinide are used to treat diabetes, which is linked to an increased risk for dementia. However, Rosiglitazone, for example, failed as a single agent in an AD clinical trial [34], suggesting different dose regimens and/or different initiation of treatment times as well as combination treatments (see below for our proposed three-drug combination).

TABLE 1

The Top Ten Repurposed Drug Candidates for AD

| Drug | Approved Treatment | Description | DeepDrug Scores |
|---|---|---|---|
| Tiludronic acid | Paget’s disease | Bisphosphonate, a regulator of calcification and decalcification. | 0.452 |
| Ziprasidone | Schizophrenia | Atypical antipsychotic | 0.422 |
| Rosiglitazone | Diabetes | Thiazolidinedione, maintains glycemic control in type 2 diabetes. | 0.383 |
| Cannabidiol | Seizures | Active cannabinoid, for the management of seizures. | 0.365 |
| Moroctocog alfa | Hemophilia A | A recombinant Factor VIII, used to treat hemophilia A to control bleeding. | 0.354 |
| Diclofenac | Osteoarthritis | non-steroidal anti-inflammatory drug (NSAID), used to treat the signs and symptoms of osteoarthritis and rheumatoid arthritis. | 0.342 |
| Sarilumab | Rheumatoid arthritis | Monoclonal antibody, used to treat moderate to severe rheumatoid arthritis. | 0.326 |
| Sulindac | Ankylosing spondylitis | NSAID, used to treat osteoarthritis, rheumatoid arthritis, ankylosing spondylitis. | 0.323 |
| Pemetrexed | Mesothelioma | Folate analog, used to treat mesothelioma and non-small cell lung cancer. | 0.320 |
| Nateglinide | Diabetes | Meglitinide, used to treat non-insulin dependent diabetes mellitus. | 0.316 |

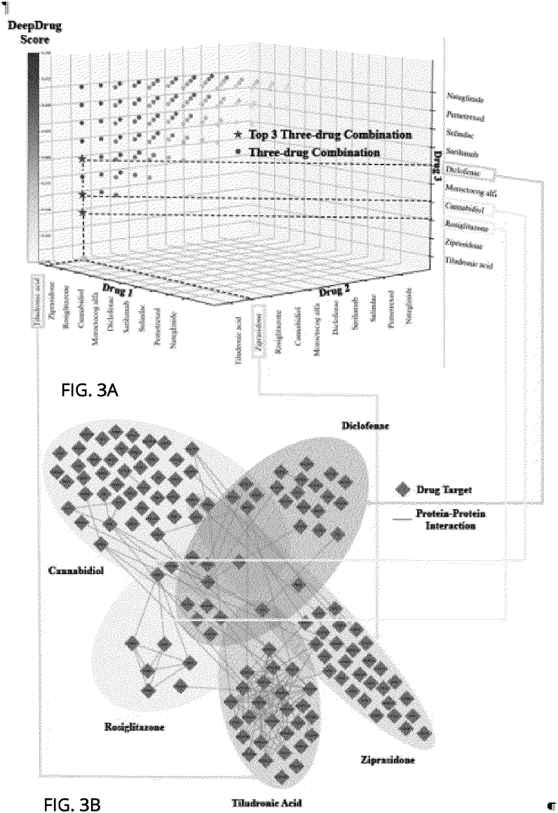

The top ten drug candidates out of an initial 749 drugs screened were used to identify the top three-drug combinations. FIG. 3 relates to DeepDrug prediction of three-drug combinations. FIG. 3A shows the 120 (ten choose three) three-drug combinations, with their DeepDrug scores indicated in color. The top ten three-drug combinations for AD are shown in Table 2. The leading three-drug combination comprised Tiludronic acid, Ziprasidone, and Cannabidiol. Based on publicly available information on DrugBank, the mechanism and structure of these three drugs are listed in Table 3. These drugs are approved to treat neurological disorders such as Schizophrenia and Seizures and bone disease (Paget’s disease) and all of them have been involved in different stages of AD drug discovery, ranging from a literature review advocating new directions in AD drug development (with Tiludronic acid 34, and with Ziprasidone 35,36) to clinical trials for treating AD patients with Cannabidiol.37

In FIG. 3A the scores of the 120 three-drug combinations (chosen from the top ten drugs) are indicated in blue, with darker colors indicating higher scores, and the top three three-drug combinations indicated by blue stars. FIG. 3B.hows the drug targets of the five drugs involved in the top 3 three-drug combinations, and the relationship between the drug targets via protein-protein interactions.

TABLE 2

The Top Ten Three-drug Combinations for AD

| Drug 1 | Drug 2 | Drug 3 | DeepDrug Scores |
|---|---|---|---|
| Tiludronic acid | Ziprasidone | Cannabidiol | 0.482 |
| Tiludronic acid | Ziprasidone | Diclofenac | 0.473 |
| Tiludronic acid | Ziprasidone | Rosiglitazone | 0.471 |
| Tiludronic acid | Ziprasidone | Moroctocog alfa | 0.471 |
| Tiludronic acid | Ziprasidone | Sarilumab | 0.471 |
| Tiludronic acid | Ziprasidone | Sulindac | 0.471 |
| Tiludronic acid | Ziprasidone | Pemetrexed | 0.471 |
| Tiludronic acid | Ziprasidone | Nateglinide | 0.471 |
| Tiludronic acid | Cannabidiol | Nateglinide | 0.463 |
| Tiludronic acid | Cannabidiol | Diclofenac | 0.463 |

TABLE 3

A Lead Three-drug Combination of FDA-approved Drugs for AD

| | | | |
|---|---|---|---|
| Drug | Tiludronic acid | Ziprasidone | Cannabidiol |
| Description | Bisphosphonate | Atypical antipsychotic | Active cannabinoid |
| Target Disease | Paget’s disease (bone disease) | Schizophrenia (brain disorder) | Seizures (brain disorder) |
| AD Drug Discovery | Literature review | Literature review | Phase-II clinical trial |
| Mechanism | Tiludronate inhibits protein-tyro sine-phosphatase, which increases tyrosine phosphorylation, and disrupts podosome formation Tiludronic acid also inhibits V-ATPases in the osteoclast, preventing F-actin from forming podosomes | Binds to serotonin-2A (5-HT2A) and dopamine D2 receptors Potent interaction with 5-HT2C, 5-HT1D, and 5-HT1A receptors in brain tissue | Act as a negative allosteric modulator of the cannabinoid CB 1 receptor |
| Structure | 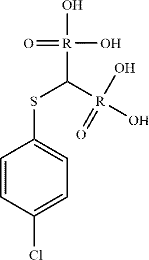 | 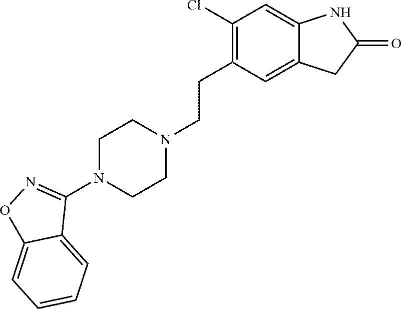 [SMILES: O=C1Cc2cc(CCN3CCN(c4noc5ccccc45)CC3)c(Cl)cc2N1] | 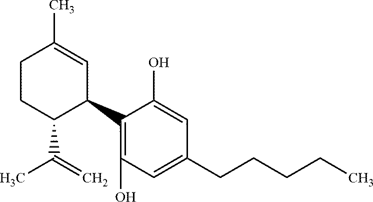 [SMILES: C=C(C)[C@@H]1CCC(C)=C[C@H]1c1c(O)cc(CCCCC)cc1O] |

The heterogeneous biomedical graph is constructed so as to incorporate domain-specific knowledge, including long genes, inflammation, immunological and aging pathways, and somatic mutation markers identified in the blood or the brain, which are closely associated with AD. Moreover, the mutation pathways causal to AD are identified based on multiple datasets representative of different AD populations, such as the Alzheimer’s Disease Neuroimaging Initiative (ADNI) dataset and the Alzheimer’s Disease Sequencing Project (ADSP) dataset, through the standard gene set enrichment analysis (GSEA). [13] Specifically, a multimodal graph is constructed comprising four types of nodes (genes, proteins, drugs, and drug targets) and four types of associations between these nodes (gene-protein, protein-protein, drug-target, and drug-drug edges). After the graph is construction isolated nodes are removed from the graph. The detailed graph construction procedures are as follows.

Gene Nodes: Two types of gene mutations relevant to AD, namely, somatic mutations and germline mutations, are considered. For somatic mutations, gene nodes are obtained from long genes [11] and somatic mutation markers are identified in the blood. [14] For germline mutations, gene nodes are obtained from the high-risk genes identified from GWAS data. [15] Other important genes relevant to (1) AD pathology (such as APP, APOE, Tau, PSEN1, PSEN2, PAX6, and ACT1), (2) healthy aging (such as SIRT1, FOXO3, and IGF1), or (3) inflammation, immunological, and aging pathways, are also selected from expert-led insights.

The gene nodes are weighted according to their chance of mutation and importance to AD progression. Data on the somatic mutations implicated in the AD pathology is obtained from Soheili-Nezhad et al. (2021) [11], constituting a total of 272 long genes. GWAS data is obtained from I. E. Jansen et al. (2019) [15] Phase 3, for a total of 30 significant genes. Ten (10) expert-led genes are chosen based on the expert knowledge, i.e., the recent discovery of ADNP and SHANK3 mutations as the key somatic mutation markers of autism, which is linked to AD. [35] In addition, it has been shown that critical somatic mutations in ADNP correlate with AD Tau pathology [28], and they have been verified as driving an early AD-like Tau pathology in a genome-edited (CRISPR/Cas9) mouse model. [36] Only two overlaps between the three sets of genes could be identified, including APOE, between GWAS and the expert-led, and CNTNAP2, between the long gene set and GWAS (see FIG. 5). Specifically, the weights of the 310 gene nodes (a total of three sets of genes) are calculated based on their lengths and normalized to [1,2]. For all GWAS genes, their normalized -log(p-value), normalized to [1, 2], are added to their existing normalized base weight based on gene length. For all expert-led genes, their existing weights (based on normalized gene length plus additional normalized weights from the GWAS step) are increased by 1, an arbitrary value that attaches the importance of these genes to the network. The arbitrary value is chosen as all values are normalized to [1,2], making it a fairly important value but not immensely large. Finally, all the gene nodes are normalized once again to [1, 2], giving the final gene node network.

TABLE 4

Weights Assigned to Drug Nodes Based on Different Combinations of Comorbidities Being Treated

| Combination of Co-morbidities Treated | Weights Assigned (Based on Odds Ratio obtained from Lin, R.C.H., et al. (2018). [38].) |
|---|---|
| Depression | 4.938 |
| Vascular Disease | 3.261 |
| Diabetes | 2.321 |
| Depression + Vascular Disease | 6.726 |
| Depression + Diabetes | 4.787 |
| Vascular Disease + Diabetes | 3.161 |
| Depression + Vascular Disease + Diabetes | 6.521 |

Additional nodes can be added at will, for example, pathways associated with post-traumatic stress syndrome (PTSD), a major risk for AD as well as pathways associated with intellectual disabilities, autism spectrum disorders and cytoskeletal function. [2] [28] The gene nodes are weighted according to their mutation type and importance to AD, such as gene length (for somatic mutations), significance level reported by GWAS (for germline mutations), or enrichment value reported by pathway analysis (for both somatic and germline mutations).

Drug Nodes: Based on FDA-approved drugs, drug nodes are selected using the top co-morbidities and risk factors related to AD as well as expert-led insights. The drug nodes are weighted according to the type and number of AD co-morbidities and risk factors.

Data on the drugs was obtained from DrugBank [18], an online database containing information on drugs and drug targets. The keywords used to find the relevant drugs are the top two risk factors of AD: 'senescence', 'female'; the top four co-morbidities of AD: 'Diabetes', `Cardiovascular disease', 'Depression', 'Inflammatory bowel disease'; miscellaneous terms: `Reactive oxygen species', `Vitamin D', 'immunological', 'autoimmune'; and the disease itself: 'Alzheimer’s'. This resulted in 1807 potential drugs. After filtering out the vaccines, imaging agents, and choosing only the FDA-approved drugs, this number was reduced to 749. The drug nodes were weighted by adding the odds ratio of the key comorbidities they treat to a default value of 1.5. Lin, R.C.H., et al. (2018) [38] reported the odds ratio of the three co-morbidities of ‘diabetes’, ‘cardiovascular disease’, and ‘depression’ in various combinations. This study, with 49747 subjects aged ≥ 65 years in 2000, was based on the Taiwan National Health Insurance Research Database, with subjects stratified by the presence and absence of dementia from 2000 to 2010. Dementia subjects (4749 out of the total cohorts) were those first diagnosed with dementia from 2000 to 2009. Table 4 shows the weights assigned to drugs that treat various combinations of these three comorbidities. The rationale being that, if a drug treats the key co-morbidities of dementia, the most prevalent form of AD, it should be at the top the search list and be accorded a higher weighting subsequently. Meanwhile, the weight of a combination of co-morbidities may not be equivalent to the sum of the individual co-morbidities. In addition, the weight of a single co-morbidity can be higher than a combination including this co-morbidity. Based on the weighting rationale, the weights of the drug-nodes were normalized to [1,2].

Target Nodes: Target nodes are obtained along with the drug nodes, as each drug has one or more specific targets. There were four types of targets in the data obtained from the DrugBank, including targets, enzymes, carriers, and transporters, resulting in 1,361 drug targets. The target nodes are weighted only by their involvement in the top AD co-morbidities and not risk factors, as it is difficult to relate proteins to risk factors. The weights of the target nodes were normalized to [1, 2].

Protein Nodes: Protein nodes are initially obtained according to the gene and target nodes, as each gene resulted in one protein and all targets were proteins in this study. Then, the first-order neighbors of the initial protein nodes are included in the graph according to the human protein-protein interaction (PPI) data. The protein nodes are weighted by the same method as that of the target nodes.

Data on the proteins are obtained from BioGRID [21], an online curated biological database. The data is first filtered to include only the proteins in human beings. Then, using the 310 proteins arising from the 310 genes, as well as the 1,361 targets, results in a total of 1,671 proteins. Next, the first-order neighbors of the 1,671 proteins are added, giving a total of 12,736 proteins. The protein nodes are weighted by their involvement in the top three co-morbidities, by adding the odds ratio of the key comorbidities they are involved in to a default value of 1.5. The weights are then normalized to [1,2].

Next, the edges between the nodes and the associated weights are determined. There are four types of edges, namely, gene-protein, drug-drug, drug-target, and protein-protein. Note that except for drug-drug edges, all other edges are directed. Descriptions on how the weights of these edges are determined can be found below, including the list and the directions of these edges, plus their associated weights.

Gene-Protein Edges: For the gene-protein edges, a unidirectional relationship between genes and proteins is established directly, as each gene leads to a single protein. The gene-protein edges are weighted uniformly and can be further weighted when mRNA stability data (i.e., how many proteins can one mRNA create before degrading) is available. With the 310 gene nodes, 310 gene-protein edges can be identified. The gene-protein edges are weighted uniformly with a default weight of 1.5, due to a lack of mRNA stability data.

Drug-Drug Edges: A default bi-directional relationship is established for the edges between the drugs. The drug-drug edges are weighted by text-mining the drug-drug interaction documents (i.e., whether the interaction of two drugs increased or decreased risk).

Data on the drug-drug interactions is obtained from the STITCH Database (‘search tool for interactions of chemicals’) [19], integrating information about the interactions from metabolic pathways, crystal structures, binding experiments, and drug-target relationships. A total of 11,728 bidirectional drug-drug edges were established. The edges are weighted using the text information of available detailed drug-drug interactions from NCBI RxNav API [20], obtained from ONCHigh and DrugBank. Specifically, a default weight of 1.5 is given and, through text-mining for the words ‘increase risk’ or ‘decrease risk’, if the interaction between two drugs is positive, 1 is added to the weight, otherwise 1 is subtracted from the weight. The weights of the drug edges ire then normalized [1, 2].

Drug-Target Edges: A unidirectional relationship between drugs and targets is established directly, as each drug has its own (one or more) specific targets. The drug-target edges are weighted uniformly, with options to further weight when drug-target efficacy data becomes available. Specifically, the drug-target edges are weighted uniformly with a default weight of 1.5, as drug-target effectiveness data was unavailable. A total of 5,310 drug-target edges were identified.

Protein-Protein Edges: A mixture of unidirectional and bidirectional relationships is established between proteins, according to the bait-hit concept, [39] allowing the PPI data to have direction, essentially leading to some edges being unidirectional. Other edges that do not have bait-hit information, are set as bidirectional by default. The protein-protein edges are weighted by calculating the protein sequence similarity of the two involved proteins, using the standard Smith-Waterman algorithm, without being normalized. A total of 89,289 protein-protein edges, either unidirectional, when the bait-hit information is available, or bidirectional, when no direction information is available, were established.

Based on the expert-led domain-specific knowledge, the weights of some protein-protein edges are further increased according to the enrichment value of the pathways if they are part of an AD-associated pathway. Specifically, pathways that overlap across multiple datasets covering different AD populations are assigned a higher score, i.e., given additional weight, with the assumption that these edges are likely to be the common denominators of AD. All genes (long, GWAS, and expert-led genes) are used to generate the AD-related pathways, using the STRING pathway analysis. [40-42].

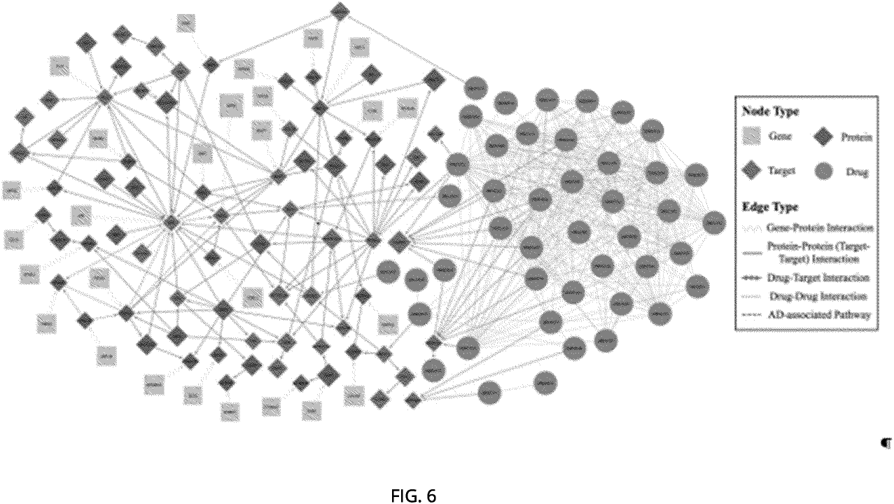

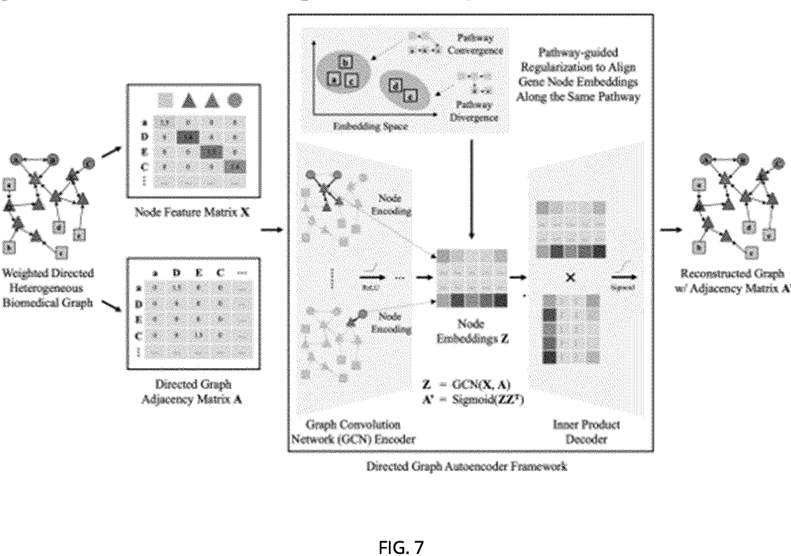

Specifically, for the top five pathways identified in the 272 long gene set and inhibited in the AD brain, [32] their -log(B-P p-values) are normalized relative to the top five to [1.5, 2.5]. The general aim was to limit the impact of pathways from being overwhelming, but also not to let it be underwhelming. Next, the not-yet-normalized PPI edge weights for those edges which contained proteins involving in a/some pathway(s) amongst those top five pathways and that exist in the PPI network of the invention, are multiplied by the normalized -log(B-P p-value) value of the corresponding pathway(s). Next, for the top ten pathways that are identified in the 30 significant GWAS gene set, their FDR values are normalized (as before, relative to the top ten) to [1.5, 2.5], and following the same logic described above, the corresponding PPI edge weights are multiplied by the normalized FDR value of the corresponding pathway(s). The protein-protein edge weights were then finally normalized to [1, 2]. FIG. 6 shows the DeepDrug directed biomedical graph. This directed biomedical graph consists of the four node types and their edges (interactions). This is a simplified version including only the top 50 nodes of each node type. This illustrates how the AD-relevant genes are connected to the proteins, then to the drug targets, and finally to the drugs.

Then, the protein-protein weight is determined according to the pathway characteristics. Upstream protein-protein edges along with pathways enriched in AD populations are assigned a higher weight to tackle the up-regulation of AD-risk genes as early as possible. Protein-protein edges along with pathways inhibited in AD populations are assigned to the same enrichment value, given that the down-regulation of any part of one pathway may lead to the abnormal functioning of one’s biological system.

Drug-Target and Drug-Drug Edges: As noted above, a unidirectional relationship between drugs and targets is established directly, as each drug has its own (one or more) specific targets. The drug-target edges are weighted uniformly and may be further weighted if drug-target effectiveness data is available. Moreover, a default bi-directional relationship is established for the edges between the drugs. The drug-drug edges are weighted by text-mining the drug-drug interaction documents (i.e., whether two drugs increased or decreased risk).

A directed GNN framework is developed to map all nodes in the heterogeneous biomedical graph, including gene and drug nodes, to the same embedding space. At a high level, the GNN framework reconstructs the graphical structure through low-dimensional node embeddings, which are calculated by a graphical convolutional network (GCN). Specifically, a variational graph autoencoder (GAE) framework [17] is adopted to determine a low-dimensional representation of each node. The GAE comprises two parts, namely, an encoder and a decoder. The encoder is a GCN, while the decoder is an inner product decoder (which can be used for unsupervised representational learning without ground truths from downstream tasks).

An adjacency matrix A^{N×N}, which represents how nodes are connected and how edges are weighted, is obtained from the multimodal graph G. In addition, a node feature matrix X^{N×4} is obtained from G, where each node is represented by a four-dimension feature vector. The four-dimension vector is the product of a node’s weight and a one-hot vector representing the node’s type (e.g., [1, 0, 0, 0] for a gene node). The encoder takes A^{N×N} and X^{N×4} as the input and produces embeddings for each node via an n-layer GCN. The decoder model optimizes low-dimensional node embeddings to reconstruct the network structure. Conceptually, the GNN framework is described as follows:

$$Z = G C N \left(X , A\right)$$

$${A}^{′} = σ \left(Z {Z}^{T}\right)$$

where
GCN is a graphical convolutional network with the ReLU activation function,
Z^{N×F} represents low-dimensional node embeddings,
σ is the sigmoid activation function,
and A′ is the edge probability matrix of all node pairs.

To optimize the GNN model parameters, the model is trained through a link prediction task on the biomedical graph. The task is a binary classification problem, which predicts the closeness between two nodes on the graph, thus allowing the user to indicate which drugs are candidates for a certain gene node. Furthermore, to account for the convergence and divergence of AD-associated pathways, a pathway-guided regularization term is incorporated into the training process. Specifically, the regularization term forces the gene nodes along the same pathway to stay closer to each other in the embedding space, while preserving the convergence and divergence information. The detailed training procedure is as follows:
1. Compute node-wise embeddings Z via an encoder
2. Compute edge probabilities based on a decoder
3. Compute the total binary cross entropy (BCE) loss for:
   a. Positive edges, which do exist in the graph
   b. Negative edges, which do not exist in the graph (generated through negative sampling in a 1:1 ratio)
4. Minimize the BCE loss while incorporating the pathway-guided regularization term to optimize the GNN parameters

To validate the GNN framework, an independent set of edges is obtained before model training by randomly removing some existing edges (positive edges) in the graph and generating some negative edges. A receiver operating characteristic (ROC) curve shows the relationship between clinical sensitivity and specificity for every possible cut-off. Two metrics are used for model evaluation, including the area under the ROC curve (AUC), which is a measure to summarize the overall prediction accuracy (true positive rate vs. true negative rate), and average precision (AP), which is the ratio of true positives and all positive predictions.

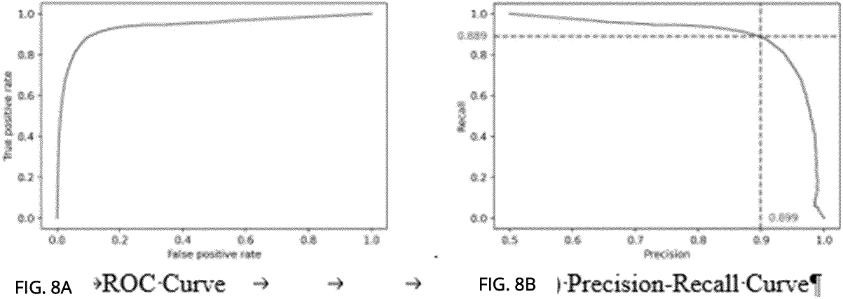

After the biomedical graph construction, a random 80/10/10 split of all existing edges on the graph is used as the training set, validation set, and test set. The embedding dimension size is selected empirically and set to 32. The Adam optimizer is used to learn the model parameters, with a learning rate of 0.01. Given that only five pathways are selected, the pathway-guided regularization term is not included in the model training process. The validation set is used for early stopping of the process during training. After model training, the optimized node embeddings are used to classify the existence of edges on the test set, and as shown in FIG. 8, achieving more than 90% classification accuracy (AUC: 0.93, AP:0.90), thus allowing for the accurate representation of the biomedical graph structure, including drug and gene nodes.

After model training and validation, all data are fed into the trained model to obtain an embedding vector for drug and gene nodes. The drug-gene score is calculated, and top drug candidates and a lead combination are selected from the top candidates (FIG. 2C). Specifically, the closer a drug’s node is to the AD-risk gene nodes, the more likely it is a higher-ranking AD drug candidate. The drug-gene score is calculated using the cosine distance in the embedding space:

$${\text{Score}}_{d , g} = 1 - \text{cosine} \left({Z}_{d} , {Z}_{g}\right)$$

where
d represents one drug,
g represents one gene,
and Z is the node embedding vector.

The top K drugs are selected based on the average drug-gene scores across the top L high-risk genes. The detailed procedure is as follows:
1. Select the top L high-risk genes via their node weights
2. Calculate the average drug-gene score for the top L high-risk genes for each drug
3. Obtain the top K drugs according to the average scores.

A lead combination is further selected that is more likely to hit AD-risk genes than others. More specifically, the average maximum drug-gene score is calculated for each drug combination among the top drugs. A lead combination of drugs (say three drugs) is selected that gives the best average maximum score. The detailed procedure is as follows:
1. Select the top K drugs
2. Exclude drugs with negative effects by domain knowledge
3. For each drug combination among the top K drugs
   a. For each of the top L high-risk genes, calculate the maximum drug-gene score from all drugs in the combination
   b. For the top L high-risk genes, calculate the average maximum drug-gene scores
4. Obtain the lead drug combination according to the average scores.
Recent developments in AI technologies, such as GNN and causal AI, as well as the accumulation of large volumes of relevant datasets such as KEGG [43], DrugBank, CMap, SPIED and DGIdb, have provided new opportunities for an AI-driven drug-repurposing approach to greatly improve the probability of finding effective AD drug candidates and vastly reducing developmental costs and accelerating precision drug identification.

Existing data-driven computational drug-repurposing methods have yet to account for expert-led AD knowledge. Moreover. it remains challenging to integrate multiple datasets representative of different AD populations to identify the mutation pathways most causal to AD, while capturing the convergence and divergence of different AD-associated pathways, in order to determine a lead combination of candidate AD drugs. To address these challenges, the present invention uses an expert-led AI-driven framework to determine a lead drug combination for AD treatments based on heterogeneous biomedical data. Despite the fact that the underlying mechanism of AD remains less well understood, the proposed GNN model of the present invention incorporates expert-led insights into the graph construction and weighting process, including long genes, inflammation, immunological and aging pathways, and somatic mutation markers identified in the blood, which are all closely associated with AD. Further, departing from previous AD drug-repurposing studies, the present invention integrates various biomedical datasets representative of different populations to identify mutation pathways causal to AD, and the convergence and divergence of AD pathways.

Capitalizing on the recent findings that long genes are more likely to be affected by somatic mutations, [11] and accumulating somatic mutations are implicated in brain pathology [28] and AD-risk blood, [14] the DeepDrug framework of the present invention has been shown capable of identifying the top ten repurposed drugs targeting long genes. In general, the identified drugs are consistent with prior knowledge. Some drugs are already approved to treat other neurodegenerative diseases, such as Parkinson’s disease, and may play an important role in AD treatment. Other drugs are approved to treat diseases that are not directly related to neurodegeneration and may need further investigation. Specifically, one drug (alpha-galactosidase A chaperone) for Fabry disease, a metabolic disorder, could be useful for other protein-folding disorders, including AD. [29] Also, one drug (xanthine oxidase inhibitor) for oxidative stress may lower the risk of AD. [30] Further, the lead drug combination, including three drugs obtained from the top drug candidates, could be a promising drug combination for AD treatments. The three drugs, covering different drug mechanisms, were originally approved to treat Parkinson’s disease and Fabry disease and have all been involved in different stages of AD drug discovery, ranging from preliminary drug review to experimental studies, including preclinical study and clinical trial.

Moreover, the demonstration of the novel DeepDrug Framework of the present invention has shown that existing approved AD drugs targeting AD symptoms tend to rank lower. None of the approved AD drugs, including the recently approved Aducanumab targeting amyloid beta (one of the AD hallmarks), were among the top ten drugs identified by the GNN model. The low rankings of approved AD drugs can be attributed to the expert-led insights incorporated into the GNN model input (which did not focus on amyloid beta only), such as long genes, co-morbidities, the importance of AD-specific somatic gene pathways. The findings from the GNN model of the present invention are also consistent with the observations from existing AD drug experiments. Until now, no drugs have been proved to treat AD effectively. The repeated failures of the approaches targeting amyloid in AD clinical trials have increasingly pointed to new potential directions in AD drug development, such as the role of neuroinflammation or immune system. [31] These results, taken together, highlight the importance of incorporating expert-led insights into the AD drug repurposing framework of the present invention.

Furthermore, the current AI-driven GNN model can be improved in three directions. First, the drug-gene relationship can be further characterized to distinguish positive drugs from negative ones. Given that no effective drug is yet available for AD, to facilitate the GNN model’s learning towards which drug can “treat” which gene, multi-task learning can be adopted to simultaneously repurpose drugs for AD and other related diseases (where ground truth drugs may be available). Second, unlike the proposed post-hoc method that identifies a lead combination among the top drug candidates, the GNN model structure can also account for the drug combinations in the first place, utilizing a hierarchical graph neural network [32] to identify different clusters of drugs while capturing the relationships of nodes at different layers through graph attention mechanisms. Third, the interpretability of the GNN model can be improved. The contribution of local subgraphs, [33] in particular, genetic pathways, in calculating the drug-gene score can be investigated to better understand the importance of different pathways in AD drug-repurposing. Such interpretations from a “black box”, combined with expert insights, will promote the feedback loop in the proposed expert-led AI-driven approach.

Finally, having identified the lead combination of drugs in silico, the test drug efficacy can be tested in vitro and in vivo. Outcome measures will confirm protection against synaptic reduction and against neuronal cell death. Further, an assessment of drug effects can be determined on β-amyloid plaque generation (by γ-secretase assay) and tau phosphorylation (by direct measurements and by assessment of the expression and activity of kinases responsible for tau phosphorylation) and effects on the formation of plaques and neurofibrillary tangles in mouse brains. Following initial in vitro screening, drug efficacy can further be tested within AD mouse models. Short-Term Spatial Memory and mouse behavior can also be tested. It is intended that the results of these studies lead to clinical trials within AD patients and that drugs identified in this study be used to effectively treat either the general AD community or specific subpopulations within it.

The DeepDrug Framework of the present invention can be applied to repurpose drugs for other neuro-degenerative and neuro-developmental diseases, including but not limited to Parkinson’s disease, autism spectrum disorders, PTSD, multiple sclerosis, and schizophrenia.

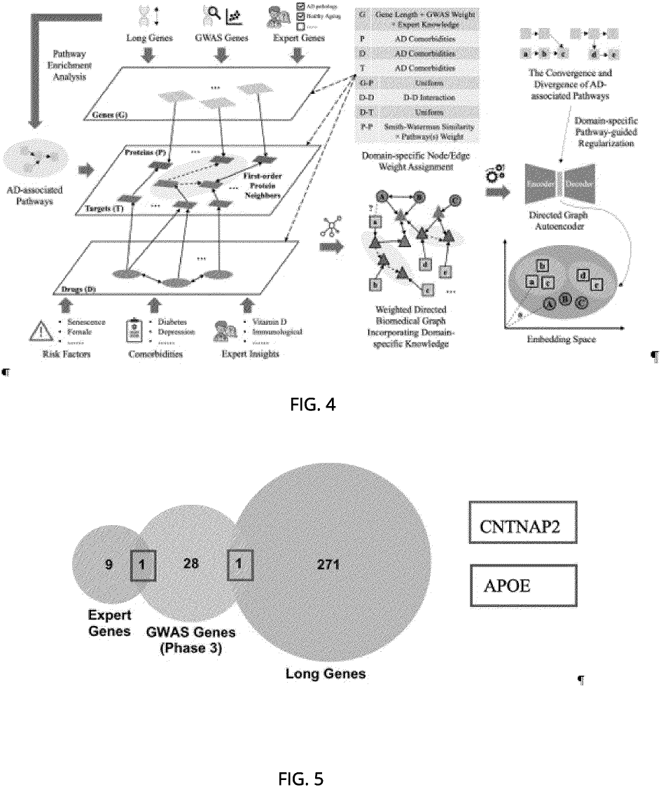

A heterogeneous directed biomedical graph was constructed by incorporating the domain-specific knowledge, including long genes, inflammation, immunological and aging pathways, and somatic mutation markers identified in the blood or the brain, which are closely associated with AD (see FIG. 4). In contrast to other biomedical graph models, the biomedical graph of the present invention allows weights to be assigned to the nodes and edges, and directions to be assigned to the edges in order to capture AD domain-specific knowledge more accurately. As inspired by Hsieh et al. (2020) [6], by encoding the present biomedical graph via GNN into a new embedding space, the complex relationship between different nodes in the original graph can be better captured than by simply using a shortest path metric. Deviating from Hsieh et al. (2020) [6], a new directed biomedical graph is developed, which is therefor novel. With the node and edge weight assignments, the present invention is capable of providing a more accurate prediction of successful drug candidates.

### References

The cited references in this application are incorporated herein by reference in their entirety and are as follows:

1. Nichols, E. et al. Global, regional, and national burden of Alzheimer’s disease and other dementias, 1990-2016: a systematic analysis for the Global Burden of Disease Study 2016. The Lancet Neurology 18, 88-106 (2019).

2. Pushpakom, S. et al. Drug repurposing: progress, challenges and recommendations. Nature Reviews Drug Discovery 18, 41-58 (2019).

3. Ekins, S. et al. Exploiting machine learning for end-to-end drug discovery and development. Nature Materials 18, 435-441 (2019).

4. Cheng, F. et al. A genome-wide positioning systems network algorithm for in silico drug repurposing. Nature Communications 10, 3476 (2019).

5. Gysi, D. M. et al. Network medicine framework for identifying drug-repurposing opportunities for COVID-19. PNAS 118, (2021).

[6. Hsieh, K. et al. Drug Repurposing for COVID-19 using Graph Neural Network with Genetic, Mechanistic, and Epidemiological Validation. Research Square rs.3.rs-114758 (2020) dot:10.21203/rs.3.rs-114758/v1.

7. Siavelis, J. C., Bourdakou, M. M., Athanasiadis, E. I., Spyrou, G. M. & Nikita, K. S. Bioinformatics methods in drug repurposing for Alzheimer’s disease. Briefings in Bioinformatics 17, 322-335 (2016).

8. Rodriguez, S. et al. Machine learning identifies candidates for drug repurposing in Alzheimer’s disease. Nature Communications 12, 1033 (2021).

9. Zeng, X. et al. deepDR: a network-based deep learning approach to in silico drug repositioning. Bioinformatics 35, 5191-5198 (2019).

10. Sims, R., Hill, M. & Williams, J. The multiplex model of the genetics of Alzheimer’s disease. Nature Neuroscience 23, 311-322 (2020).

11. Soheili-Nezhad, S., Linden, R. J. van der, Rikkert, M. O., Sprooten, E. & Poelmans, G. Long genes are more frequently affected by somatic mutations and show reduced expression in Alzheimer’s disease: Implications for disease etiology. Alzheimer’s & Dementia 17, 489-499 (2021).

12. Park, J. S. et al. Brain somatic mutations observed in Alzheimer’s disease associated with aging and dysregulation of tau phosphorylation. Nature Communications 10, 3090 (2019).

13. Subramanian, A. et al. Gene set enrichment analysis: A knowledge-based approach for interpreting genome-wide expression profiles. PNAS 102, 15545-15550 (2005).

14. Sragovich, S., Gershovits, M., Lam, J. C. K., Li, V. O. K. & Gozes, I. Putative Blood Somatic Mutations in Post-Traumatic Stress Disorder-Symptomatic Soldiers: High Impact of Cytoskeletal and Inflammatory Proteins. Journal of Alzheimer’s Disease 79, 1723-1734 (2021).

15. Jansen, I. E. et al. Genome-wide meta-analysis identifies new loci and functional pathways influencing Alzheimer’s disease risk. Nature Genetics 51, 404-413 (2019).

16. Bader, G. D. & Hogue, C. W. V. Analyzing yeast protein-protein interaction data obtained from different sources. Nat Biotechnol 20, 991-997 (2002).

17. Kipf, T. N. & Welling, M. Variational Graph Auto-Encoders. arXiv:1611.07308 [cs, stat] (2016).

18. Wishart, D. S. et al. DrugBank: a comprehensive resource for in silico drug discovery and exploration. Nucleic Acids Research 34, D668-D672 (2006).

19. Kuhn, M. et al. STITCH 2: An interaction network database for small molecules and proteins. Nucleic Acids Research 38, D552-556 (2010).

20. Drug Interaction API. https://rxnav.nlm.nih.gov/InteractionAPIs.html.

21. Oughtred, R. et al. The BioGRID database: A comprehensive biomedical resource of curated protein, genetic, and chemical interactions. Protein Science 30, 187-200 (2021).

22. Torres-Bondia, F. et al. Proton pump inhibitors and the risk of Alzheimer’s disease and non-Alzheimer’s dementias. Scientific Reports 10, 21046 (2020).

23. Hsu, J.-Y. et al. Lower Risk of Dementia in Patients With Atrial Fibrillation Taking Non-Vitamin K Antagonist Oral Anticoagulants: A Nationwide Population-Based Cohort Study. Journal of the American Heart Association 10, e016437 (2021).

24. Alexander, G. C., Emerson, S. & Kesselheim, A. S. Evaluation of Aducanumab for Alzheimer Disease: Scientific Evidence and Regulatory Review Involving Efficacy, Safety, and Futility. JAMA 325, 1717-1718 (2021).

25. Bonam, S. R., Wang, F. & Muller, S. Lysosomes as a therapeutic target. Nature Reviews Drug Discovery 18, 923-948 (2019).

26. Park, J.-H. et al. Newly developed reversible MAO-B inhibitor circumvents the shortcomings of irreversible inhibitors in Alzheimer’s disease. Science Advances 5, eaav0316 (2019).

27. Matthews, D. C. et al. Rasagiline effects on glucose metabolism, cognition, and tau in Alzheimer’s dementia. Alzheimer’s & Dementia: Translational Research & Clinical Interventions 7, e12106 (2021).

28. Ivashko-Pachima, Y. et al. Discovery of autism/intellectual disability somatic mutations in Alzheimer’s brains: mutated ADNP cytoskeletal impairments and repair as a case study. Molecular Psychiatry 26, 1619-1633 (2021).

29. Guce, A. I., Clark, N. E., Rogich, J. J. & Garman, S. C. The Molecular Basis of Pharmacological Chaperoning in Human α-Galactosidase. Chemistry & Biology 18, 1521-1526 (2011).

30. Chuang, T.-J., Wang, Y.-H., Wei, J. C.-C. & Yeh, C.-J. Association Between Use of Anti-gout Preparations and Dementia: Nested Case-Control Nationwide Population-Based Cohort Study. Frontiers in Medicine 7, 607808 (2020).

31. Mullane, K. & Williams, M. Alzheimer’s disease beyond amyloid: Can the repetitive failures of amyloid-targeted therapeutics inform future approaches to dementia drug discovery? Biochemical Pharmacology 177, 113945 (2020).

32. Ying, R. et al. Hierarchical Graph Representation Learning with Differentiable Pooling. arXiv:1806.08804 [cs, stat] (2019).

33. Ying, R., Bourgeois, D., You, J., Zitnik, M. & Leskovec, J. GNNExplainer: Generating Explanations for Graph Neural Networks. arXiv:1903.03894 [cs, stat] (2019).

34. Gold, M. et al. Rosiglitazone monotherapy in mild-to-moderate Alzheimer’s disease: results from a randomized, double-blind, placebo-controlled phase III study. Dementia and Geriatric Cognitive Disorders 30, 131-146 (2010).

35. Ivashko-Pachima, Y. et al. SH3-and actin-binding domains connect ADNP and SHANK3, revealing a fundamental shared mechanism underlying autism. Molecular Psychiatry, 1-12 (2022).

36. Karmon, G. et al. Novel ADNP syndrome mice reveal dramatic sex-specific peripheral gene expression with brain synaptic and Tau pathologies. Biological Psychiatry (2021).

37. Prince, M. J. et al. World Alzheimer Report 2015-The Global Impact of Dementia: An analysis of prevalence, incidence, cost and trends. (2015).

38. Lin, C. H. R. et al. Quantitative comorbidity risk assessment of dementia in Taiwan: A population-based cohort study. Medicine 97 (2018).

39. Ballard, C. et al. Drug repositioning and repurposing for Alzheimer disease. Nature Reviews Neurology 16, 661-673 (2020).

40. Mering, C. v. et al. STRING: A database of predicted functional associations between proteins. Nucleic Acids Research 31, 258-261 (2003).

41 Szklarczyk, D. et al. STRING v11: Protein-protein association networks with increased coverage, supporting functional discovery in genome-wide experimental datasets. Nucleic Acids Research 47, D607-D613 (2019).

42 STRING. STRING, <https://string-db.org/> (2022).

43 KEGG. KEGG PATHWAY Database, <https://www.genome.jp/kegg/pathway.html> (2022).

While the invention is explained in relation to certain embodiments, it is to be understood that various modifications thereof will become apparent to those skilled in the art upon reading the specification. Therefore, it is to be understood that the invention disclosed herein is intended to cover such modifications as fall within the scope of the appended claims.

1. An artificial intelligence (AI)-driven drug-repurposing method to identify a lead combination of previously FDA-approved drugs to treat a disease, comprising the steps of:
constructing a heterogeneous biomedical graph comprising complex and interconnected genes, proteins, and drug information to capture the network characteristics of the disease pathology, considering expert known associations and overlaps between different disease pathways, and utilizing node weighting and edge weighting and direction;
using the graph as an input to an artificial intelligence (AI)-driven graphical neural network (GNN) framework, with embeddings of drug and gene nodes as the outputs; and
conducting a drug scoring and selection analysis on the outputs of the GNN framework to generate drug-gene scores and identify a lead combination of repurposed disease drug candidates for clinical verification.

2. The AI-driven drug-repurposing method of claim 1 wherein the disease is a neuro-degenerative or neuro-developmental disease.

3. The AI-driven drug-repurposing method of claim 2 wherein the disease is one of Alzheimer’s disease, Parkinson’s disease, autism spectrum disorders, PTSD, multiple sclerosis, and schizophrenia.

4. The AI-driven drug-repurposing method of claim 1 wherein the disease is Alzheimer’s disease (AD).

5. The AI-driven drug-repurposing method of claim 1 wherein the expert known associations include key domain-specific knowledge, such as long genes, inflammation, immunological and aging pathways, and somatic mutation markers identified from the blood and the brain.

6. The AI-driven drug-repurposing method of claim 1 wherein the step of constructing a heterogeneous biomedical graph involves integrating multiple datasets representative of different populations of the disease to identify the mutation pathways that cause the disease.

7. The AI-driven drug-repurposing method of claim 1 wherein the step of conducting a drug scoring and selection analysis involves selecting drugs that interacts with the somatic mutation phenotypes of the disease, directly or indirectly through network-based actions.

8. The AI-driven drug-repurposing method of claim 1 wherein the step of constructing a heterogeneous biomedical graph captures the convergence and divergence of different pathways that associate with the disease.

9. The AI-driven drug-repurposing method of claim 1 wherein the step of constructing a heterogeneous biomedical graph involves
constructing a multimodal graph comprising four types of nodes (genes, proteins, drugs, and drug targets) and four types of associations between these nodes (gene-protein, protein-protein, drug-target, and drug-drug edges); and
removing isolated nodes from the graph after it is constructed.

10. The AI-driven drug-repurposing method of claim 4 wherein the step of constructing a heterogeneous biomedical graph involves
constructing a multimodal graph comprising four types of nodes (genes, proteins, drugs, and drug targets) and four types of associations between these nodes (gene-protein, protein-protein, drug-target, and drug-drug edges), and
removing isolated nodes from the graph after it is constructed; and
wherein the gene nodes are somatic mutations obtained from long genes and whose markers are identified in blood, germline mutations obtained from high-risk genes identified from GWAS data, and genes relevant to (1) AD pathology (such as APP, APOE, Tau, PSEN1, PSEN2, PAX6, and ACT1), (2) healthy aging (such as SIRT1, FOXO3, and IGF1), or (3) inflammation, immunological, and aging pathways.

11. The AI-driven drug-repurposing method of claim 10 wherein during the step of constructing a heterogeneous biomedical graph the gene nodes are weighted according to their mutation type and importance to AD, such as gene length (for somatic mutations), significance level reported by GWAS (for germline mutations), or enrichment value reported by pathway analysis (for both somatic and germline mutations).

12. The AI-driven drug-repurposing method of claim 10 wherein during the step of constructing a heterogeneous biomedical graph the drug nodes are selected using the top co-morbidities and risk factors related to AD as well as expert-led insights, and the drug nodes are weighted according to the type and number of AD co-morbidities and risk factors.

13. The AI-driven drug-repurposing method of claim 12 wherein during the step of constructing a heterogeneous biomedical graph the target nodes are obtained along with the drug nodes, as each drug has one or more specific targets, and the target nodes are weighted only by their involvement in the top AD co-morbidities.

14. The AI-driven drug-repurposing method of claim 13 wherein during the step of constructing a heterogeneous biomedical graph the protein nodes are initially obtained according to the gene and target nodes, as each gene resulted in one protein and all targets were proteins; and
wherein first-order neighbors of initial protein nodes are included in the graph according to human protein-protein interaction (PPI) data and the protein nodes are weighted by the same method as the target data.

15. The AI-driven drug-repurposing method of claim 10 wherein during the step of constructing a heterogeneous biomedical graph the gene-protein edges are directly established by a unidirectional relationship between genes and proteins, as each gene leads to a single protein, and
wherein the gene-protein edges are weighted uniformly and can be further weighted if mRNA stability data (i.e., how many proteins can one mRNA create before degrading) is available.

16. The AI-driven drug-repurposing method of claim 10 wherein during the step of constructing a heterogeneous biomedical graph the protein-protein edge is established by a mixture of unidirectional and bidirectional relationships between proteins, according to the bait-hit concept, allowing the PPI data to have direction, essentially leading to some edges being unidirectional and, for other edges which do not have bait-hit information, being default bidirectional,
wherein the protein-protein edges are weighted by calculating the protein sequence similarity of the two involved proteins, using the standard Smith-Waterman algorithm; and
wherein the weights of some protein-protein edges are further increased according to the enrichment value of the pathways if they were part of AD-associated pathways, specifically
pathways that overlap across multiple datasets covering different AD populations are assigned a higher score, because they are likely to be the common denominators of AD,
then, the protein-protein weight is determined according to the pathway characteristics,
upstream protein-protein edges along with pathways enriched in AD populations are assigned a higher weight to tackle the up-regulation of AD-risk genes as early as possible, and
protein-protein edges along with pathways inhibited in AD populations are assigned to the same enrichment value, given that the down-regulation of any part of one pathway may lead to the abnormal functioning of one’s biological system.

17. The AI-driven drug-repurposing method of claim 10 wherein during the step of constructing a heterogeneous biomedical graph the drug-target and drug-drug edges are established directly by a unidirectional relationship between drugs and targets, as each drug has its own (one or more) specific targets; and
wherein the drug-target edges are weighted uniformly and can be further weighted when drug-target effectiveness data is available;
a default bi-directional relationship is established for the edges between the drugs and the drug-drug edges are weighted by text-mining the drug-drug interaction documents (i.e., whether two drugs increased or decreased risk).

18. The AI-driven drug-repurposing method of claim 1 wherein GNN framework maps all nodes in the heterogeneous biomedical graph, including gene and drug nodes, to the same embedding space through low-dimensional node embeddings, which are calculated by a graphical convolutional network (GCN) with a variational graph autoencoder (GAE) framework adopted to determine a low-dimensional representation of each node, the GAE comprises an encoder and a decoder where the encoder is a GCN and the decoder is an inner product decoder.

19. The AI-driven drug-repurposing method of claim 1 wherein the decoder can be used for unsupervised representational learning without ground truths from downstream tasks.

20. The AI-driven drug-repurposing method of claim 1 wherein the GNN model is trained through a link prediction binary classification task on the biomedical graph with a pathway-guided regularization term that forces the gene nodes along the same pathway to stay closer to each other in the embedding space, while preserving the convergence and divergence information, wherein the training comprises the steps of:
1. computing node-wise embeddings Z via an encoder,
2. computing edge probabilities based on a decoder, and
3. computing the total binary cross entropy (BCE) loss for:
a. positive edges, which do exist in the graph,
b. negative edges, which do not exist in the graph (generated through negative sampling in a 1:1 ratio).

21. The AI-driven drug-repurposing method of claim 1 wherein selection analysis on the outputs of the GNN framework selects the top K drugs based on the average drug-gene scores across the top L high-risk genes as follows.
1. select the top L high-risk genes via their node weights,
2. calculate the average drug-gene score for the top L high-risk genes for each drug, and
3. obtain the top K drugs according to the average scores.

22. The AI-driven drug-repurposing method of claim 1 wherein a lead combination of repurposed disease drug candidates is selected by taking the average maximum drug-gene score calculated for each drug combination among the top drugs as follows:
1. select the top K drugs
2. exclude drugs with negative effects by domain knowledge
3. for each drug combination among the top K drugs
a. for each of the top L high-risk genes, calculate the maximum drug-gene score from all drugs in the combination,
b. for the top L high-risk genes, calculate the average maximum drug-gene scores; and
4. obtain the lead drug combination according to the average scores.

23. The AI-driven drug-repurposing method of claim 1 wherein the lead candidates for AD are Safinamide, Tecovirimat, Esomeprazole, Warfarin, Rasagiline, Silicon, Glucose, Migalastat, Allopurinol, Fingolimod, Voxelotor and Selexipag.

24. Treating a patient with symptoms or precursor signs of AD with one or more of an effective dose of Safinamide, Tecovirimat, Esomeprazole, Warfarin, Rasagiline, Silicon, Glucose, Migalastat, Allopurinol, Fingolimod, Voxelotor and Selexipag.

25. The AI-driven drug-repurposing method of claim 23 wherein the lead candidates for AD are Safinamide, Rasagiline and Migalastat.

26. The method of treating a patient according to claim 24 wherein an effective dose of one or more of Safinamide, Rasagiline and Migalastat is administered.
